(19) United States
(12) Patent Application Publication
(10) Pub. No.: US 2018/0110978 A1
(43) Pub. Date: Apr. 26, 2018

(54) SYNERGISTIC REGULATED CELL DEATH INDUCTION WITH HSP90 INHIBITORS AND NANOSECOND PULSED ELECTRIC FIELDS

(71) Applicant: Old Dominion University Research Foundation, Norfolk, VA (US)

(72) Inventors: Stephen J. BEEBE, Norfolk, VA (US); John D. CATRAVAS, Virginia Beach, VA (US)

(73) Assignee: Old Dominion University Research Foundation, Norolk, VA (US)

(21) Appl. No.: 15/559,324

(22) PCT Filed: Mar. 18, 2016
(86) PCT No.: PCT/US2016/023043

§ 371 Date: Sep. 18, 2017

Related U.S. Application Data
(60) Provisional application No. 62/135,466, filed on Mar. 19, 2015.

(51) Int. Cl. A61N 1/36; A61K 31/395; A61K 45/06; A61P 35/00

(52) U.S. Cl. CPC A61N 1/36002; A61P 35/00; A61K 45/06; A61K 31/395

(57) ABSTRACT

Methods for treating tumors employing HSp90 inhibitors in combination with nanosecond pulsed electric fields (nsPEFs) are disclosed. The methods are directed to induce regulated cell death (RCD) in tumor cells and tissues. Further, Hsp90 inhibitors in combination with nsPEF are used at low non-toxic concentrations, thereby reducing the side-effects associated with these drugs. Additionally, nsPEFs are employed at lower electric fields and/or with fewer number of pulses than when nsPEFs are employed alone. Further, the mechanisms by which nsPEFs and Hsp90 inhibitors act upon cancer cells are different, thereby combining these treatments results in a synergistic effect.

### CROSS-REFERENCE TO RELATED APPLICATIONS

This application claims priority to U.S. Provisional Application Ser. No. 62/135,466, filed Mar. 19, 2015, which is hereby incorporated by reference.

### BACKGROUND

### Field of the Disclosure

The present disclosure relates generally to methods for the treatment of tumors, and more particularly, to induction of regulated cell death of cancer cells employing Hsp90 inhibitors in combination with nanosecond pulsed electric fields (nsPEFs).

### Background Information

A neoplasm, or tumor, is an abnormal, unregulated, and disorganized proliferation of cell growth, and is generally referred to as cancer. A neoplasm is malignant, or cancerous, if it has properties of destructive growth, invasiveness, and metastasis. Cancer is now primarily treated with one or more of the three types of conventional therapies: surgery, radiation, and chemotherapy. Surgery involves the bulk removal of diseased tissue. While surgery is sometimes effective in removing tumors located at certain sites, such as, for example in the breast, colon, and skin, it cannot be used in the treatment of tumors located in other areas, such as the backbone, nor in the treatment of disseminated neoplastic conditions, such as leukemia. Radiation therapy involves the exposure of living tissue to ionizing radiation causing death or damage to the exposed cells. Side-effects from radiation therapy may be acute and temporary, while others may be irreversible. Chemotherapy involves the disruption of cell replication or cell metabolism. It is used most often in the treatment of breast, lung, and testicular cancers.

The adverse effects of systemic chemotherapy employed in the treatment of neoplastic diseases are most feared by patients undergoing treatment for cancer. Of these adverse effects, nausea and vomiting are the most common. Other adverse side-effects include cytopenia; infection; cachexia; mucositis in patients receiving high doses of chemotherapy with bone marrow rescue or radiation therapy; alopecia (hair loss); cutaneous complications, such as, for example pruritus, urticaria, and angioedema; neurological complications; pulmonary and cardiac complications; and reproductive and endocrine complications. Chemotherapy-induced side-effects significantly impact the quality of life of the patient and may dramatically influence patient compliance with treatment. As such, improved methods of treatment are needed.

In recent years, there has been a transition to treatments that are targeted so as to avoid the adverse effects of conventional cancer therapies. In this regard, several proteins have been identified as potential targets for cancer treatment. Among identified proteins, Hsp90 is one of the most abundant cytosolic proteins in cells. Hsp90 is involved in folding, stability, activation, function and assembly of several proteins, known as Hsp90 client proteins. Hsp90 is overexpressed in many cancers [Tian, W-L., He. F., Fu, X., Lin, J-T., Tang, P., Huang, Y-M., Guo, R., and Sun, L., 2014. High expression of heat shock protein 90 alpha and its significance in human acute leukemia cells. Gene, 542(2), pp. 122-128] and has many oncoproteins as its clients. Inhibitors of Hsp90 have been employed as therapeutic agents in cancer treatment [Neckers, L., Schulte, T. W. and Mimnaugh, E., 1999. Geldanamycin as a—potential anti-cancer agent: its molecular target and biochemical activity. Investigational new drugs, 17(4), pp. 361-373; Kabakov, A. E., Kudryavtsev, V. A. and Gabai, V. L., 2010. Hsp90 inhibitors as promising agents for radiotherapy. Journal of molecular medicine, 88(3), pp. 241-247], and Hsp90 has been shown to sensitize cells to a number of drugs [Nagaraju, G. P., Alese, O. B., Landry, J., Diaz, R. and El-Rayes, B. F., 2014. Hsp90 inhibition downregulates thymidylate synthase and sensitizes colorectal cancer cell lines to the effect of 5FU-based chemotherapy. Oncotarget, 5(20), pp. 9980] and radiation [Wang, B., Chen, L., Ni, Z., Dai, X., Qin, L., Wu, Y., Li, X., Xu, L., Lian, J. and He, F., 2014. Hsp90 inhibitor 17-AAG sensitizes Bcl-2 inhibitor (−)-gossypol by suppressing ERK-mediated protective autophagy and Mcl-1 accumulation in hepatocellular carcinoma cells. Experimental cell research, 328(2), pp. 379-387].

Other approaches have employed electric fields in several different types of cancer therapy. Some involve radio frequency or microwave devices that heat the tumor to greater than 43° C. to kill the cells via hyperthermia [Tanabe, K. K., Curley, S. A., Dodd, G. D., Siperstein, A. E. and Goldberg, S. N., 2004. Radiofrequency ablation. Cancer, 100(3), pp. 641-650; Haemmerich, D. and Laeseke, P. F., 2005. Thermal tumour ablation: devices, clinical applications and future directions. Int. J. Hyperthermia, 21(8), pp. 755-760]. Further approaches use pulsed electric fields to electroporate the tumor cells to allow the introduction of toxic drugs or DNA [Lucas, M. L. and Heller, R., 2003. IL-12 gene therapy using an electrically mediated nonviral approach reduces metastatic growth of melanoma. DNA and cell biology, 22(12), pp. 755-763; Kubota, Y., Tomita, Y., Tsukigi, M., Kurachi, H., Motoyama, T. and Mir, L. M., 2005. A case of perineal malignant melanoma successfully treated with electrochemotherapy. Melanoma research, 15(2), pp. 133-134; Gothelf, A., Mir, L.M. and Gehl, J., 2003. Electrochemotherapy: results of cancer treatment using enhanced delivery of bleomycin by electroporation. Cancer treatment reviews, 29(5), pp. 371-387].

More recently, nanosecond pulsed electric fields (nsPEFs) have been tested as an alternative cancer electrotherapy for different types of cancer, such as fibrosarcoma tumors. When these tumors were treated in situ with nsPEFs, exhibited a reduced growth rate compared to control tumors in the same animal [Beebe, S. J., Fox, P. M., Rec, L. J., Somers, K., Stark, R. H. and Schoenbach, K. H., 2002. Nanosecond pulsed electric field (nsPEF) effects on cells and tissues: apoptosis induction and tumor growth inhibition. Plasma Science, IEEE Transactions on, 30(1), pp. 286-292]. Further, conventional electroporation primarily affects plasma membranes whereas nsPEFs electroporate intracellular membranes as well, thereby affecting intracellular structures and functions.

Although modern cancer treatments are reasonably efficient, they are not as safe and effective as needed and thereby there still is a widely recognized need for more efficient, more specific and safer methods to eliminate cancer cells without the serious side-effects caused by conventional cancer treatments.

### SUMMARY

One or more aspects of the present disclosure provide methods for inducing regulated cell death (RCD) in tumor cells and tissues by employing one or more Hsp90 inhibitors in combination with nanosecond pulsed electric fields (nsPEFs).

In some embodiments, drugs that inhibit the specific chaperone protein Hsp90 are employed in combination with nsPEFs to induce regulated cell death (RCD) in tumors and tissues, thereby enabling elimination of cancer cells. In these embodiments, Hsp90 inhibitors in combination with nsPEF are employed at low non-toxic concentrations, thereby reducing the side-effects associated with aforementioned drugs. Further to these embodiments, nsPEFs are employed at lower electric fields and/or with fewer number of pulses than when nsPEFs are employed alone. Still further to these embodiments, the mechanism by which nsPEFs and Hsp90 inhibitors act upon cancer cells are different, thereby combining these treatments result in a synergistic effect. In these embodiments, this synergistic effect significantly eliminates cancer cells more effectively and with fewer side-effects than when either treatment is employed alone.

In some embodiments, Hsp90 inhibitors employed in combination with nsPEFs include 17-AAG, 17-DMAG, retaspimycin, macbecin, CNF-2024, CNF-1010, AT-13387, PF-04928473, STA-9090, AUY922, IPI-504, IPI-493, CCT018159, VER-49009, among others. In these embodiments, other means for inhibiting Hsp90, other Hsp90 inhibitors currently available, and gene-targeting approaches (e.g., siRNA, shRNA, CRISP against Hsp90, dominant negative or otherwise mutated Hsp90, and the like) can also be employed. Further to these embodiments, the electric pulses employed to generate nsPEFs employ a pulse duration from about 1 ns to about 1000 ns with amplitudes ranging from about 0.01 kV to about 300 kV. In an example, a single 600 ns pulse with an amplitude range from about 0 to about 80 kV/cm is employed.

In some embodiments, Hsp90 inhibitors inhibit HIF1a, which promotes expression of VEGF and vascularization of tumors. In these embodiments, nsPEFs also inhibit expression of VEGF as well as other endothelial cell markers, such as, for example CD31, CD35 and CD105, among others. Further to these embodiments, the combination of Hsp90 inhibitors and nsPEFs exhibit a synergistic effect to prevent vascularization in addition to the synergy of anti-tumor activities.

In some embodiments, the combination of Hsp90 inhibitor and nsPEF is administered alone as the sole therapeutic agent. In other embodiments, the combination of Hsp90 inhibitor and nsPEF is employed in combination with one or more additional therapies, such as, for example radiation therapy, electrotherapy, and immunotherapy, among others. In further embodiments, other anti-cancer drugs can be employed in combination with Hsp90 inhibitors and nsPEFs. In some embodiments, treatment methods employing a combination of Hsp90 inhibitors and nsPEFs can be used for treatment of tumors of different types, sizes, and at different tumor stages. In an example, treatment methods employing a combination of Hsp90 inhibitors and nsPEFs is used for treating abnormal or benign growths, such as, for example in breast (adenosis, fibroadenomas mastitis) and lungs (harmartoma, papilloma), among others.

In some embodiments, Hsp90 inhibitors are delivered to the patient before the application of nsPEFs. In other embodiments, nsPEFs are first applied to cancer cells or tumors as a sensitizer for the subsequently treatment with Hsp90 inhibitors. In these embodiments, Hsp90 inhibitors can be administered using different routes of administration, such as, for example intravenous administration (IV), oral administration, intratumoral or intraperitoneal injection, and the like.

In some embodiments, a method of inducing regulated cell death in a target tissue or target cell population is provided. The method may comprise inhibiting Hsp90 in the target tissue or target cell population, and applying an electrical stimulation to the target tissue or target cell population, the electrical stimulation comprising one or more electric pulses having a pulse duration from about 1 nanoseconds to about 1,000 nanoseconds, and further having pulse amplitudes ranging from about 0.01 kV to about 300 kV, in a synergistic manner effective to induce tissue or cell death in the target tissue or target cell population near an applied electric field created by the electrical stimulation. The target tissue may comprise a tumor, while the target cell population may comprise cancer cells. The levels of Hsp90 within the target tissue or target cell population may be over-expressed.

According to one aspect of the method, the step of inhibiting may comprise administering at least one Hsp90 inhibitor to the target tissue or target cell population. In some embodiments, the at least one Hsp90 inhibitor may be selected from the group consisting of 17-AAG, 17-DMAG, retaspimycin, macbecin, CNF-2024, CNF-1010, AT-13387, PF-04928473, STA-9090, AUY922, IPI-504, IPI-493, CCT018159, and VER-49009. According to another aspect of the method, the at least one Hsp90 inhibitor may be administered by intravenous administration, oral administration, intratumoral or interaperitoneal injection. According to still another aspect of the method, the step of inhibiting Hsp90 may employ a gene-targeted expression or promotion technique comprising siRNA, ShRNA, CRISP against Hsp90, or dominant negative or mutated Hsp90.

In some embodiments, the method may further include preventing vascularization in the target tissue or target cell population. Still, in some embodiments, the method may further include inhibiting expression of VEGF, CD31, CD36 or CD105.

In some embodiments, the step of inhibiting Hsp90 may occur before the step of applying electrical stimulation. Still, in some embodiments, the step of inhibiting Hsp90 may occur after the step of applying electrical stimulation.

In some embodiments, the method may further include the step of administering an additional treatment technique comprising radiation therapy, electrotherapy, or immunotherapy. Still, in some embodiments, the method may further include the step of administering an anti-cancer drug to the target tissue or target cell population.

According to another aspect of the method, the step of applying electrical stimulation may comprise placing electrodes into the target tissue or the target cell population in proximity to cancer cells. In some embodiments, the at least one electric pulse may comprise a pulse duration of about 600 ns. Still, in some embodiments, the at least one electric pulse may comprise an amplitude of between about 10 kV/cm to about 80 kV/cm.

In summary, the combination of Hsp90 inhibitors and nsPEFs provide a synergistic induction of RCD by different mechanisms in cancer cells. Further, Hsp90 inhibitors increase caspase 3/7 activity and consequently apoptotic cell death, whereas nsPEF induces influxes of Ca²⁺, dissipation of ΔΨm, and RCD by a caspase-independent mechanism(s). Additionally, Hsp90 inhibitors sensitize cells to nsPEFs and the combination of both treatments is a significant improvement over either treatment employed alone. Therefore, it is possible to employ low non-toxic doses of 17-AAG or other Hsp90 inhibitor and low “doses” of nsPEFs by decreasing electric field intensities or decreasing the number of pulses for treatment of cancer cells. The combination of nsPEFs and an Hsp90 inhibitor can be employed to effectively kill cancer cells while exhibiting a reduction of the side-effects associated with both treatments.

### BRIEF DESCRIPTION OF THE DRAWINGS

FIG. 1 is a graphical representation illustrating a synergistic induction of cell death in N1-S1 rat HCC cells employing nanosecond pulsed electric fields (nsPEFs) and Hsp90 inhibitor (e.g., 17-AAG), according to an embodiment.

FIG. 2 is a graphical representation illustrating effects of the nsPEFs on caspase 3/7 catalytic activity as compared to a control, according to an embodiment.

FIG. 3 is a graphical representation illustrating effects of the nsPEFs on mitochondria membrane potential (ΔΨm), Ca²⁺ influx, and cell viability in N1-S1 HCC cells, according to an embodiment.

FIG. 4 is a graphical representation illustrating effects of the Hsp90 inhibitor (e.g., 17-AAG) on caspase 3/7 catalytic activity in N1-S1 cells at different incubation times, according to an embodiment.

FIG. 5 is a graphical representation illustrating effects on cell viability of an Hsp90 inhibitor (e.g., 17-AAG) in combination with an nsPEF, according to an embodiment.

FIG. 6 is a graphical representation illustrating an additional percentage reduction in cell viability when employing an Hsp90 inhibitor at a concentration of about 0.3 μM (e.g., 17-AAG) in combination with nsPEFs at different electric field amplitudes, according to an embodiment.

FIG. 7 is a graphical representation illustrating western blot test results for cleaved caspase 3 when employing an Hsp90 inhibitor at different concentrations, according to an embodiment.

FIG. 8 is a graphical representation illustrating western blot test results for cleaved caspase 3 when employing an Hsp90 inhibitor alone, nsPEF alone, or a combination of both treatments, according to an embodiment.

### DETAILED DESCRIPTION

The present invention is described with reference to the attached figures, wherein like reference numerals are used throughout the figures to designate similar or equivalent elements. Several aspects of the invention are described below with reference to example applications for illustration. It should be understood that numerous specific details, relationships, and methods are set forth to provide a full understanding of the disclosure. One having ordinary skill in the relevant art, however, will readily recognize that the embodiments of the disclosure can be practiced without one or more of the specific details or with other methods. In other instances, well-known structures or operations are not shown in detail to avoid obscuring the aspects of the disclosure. The present disclosure is not limited by the illustrated ordering of acts or events, as some acts may occur in different orders and/or concurrently with other acts or events. Furthermore, not all illustrated acts or events are required to implement a methodology in accordance with the present disclosure.

Definitions

As used here, the following terms have the following definitions:

“Mitochondrial membrane potential (ΔΨm)” refers to a parameter of mitochondrial function that acts as an indicator that the cells will be able to convert oxygen to cellular energy.

“Nanosecond pulsed electric fields (nsPEFs)” refers to electric pulses of nanosecond duration.

Description of the Disclosure

For the purposes of promoting an understanding of the principles of the disclosure, reference will now be made to preferred embodiments and specific language will be used to describe the same. It will nevertheless be understood that no limitation of the scope of the disclosure is thereby intended. Rather, such alterations and further modifications of the disclosure, and such further applications of the principles of the disclosure as illustrated herein, as would be contemplated by one having skill in the art to which the disclosure relates are intended to be part of the present disclosure.

For example, features illustrated or described as part of one embodiment can be used on other embodiments to yield a still further embodiment. Additionally, certain features may be interchanged with similar devices or features not mentioned yet which perform the same or similar functions. It is therefore intended that such modifications and variations are included within the totality of the present disclosure.

In view of the limitations of existing methods to treat tumors, the various embodiments in the present disclosure are directed to a new method to induce regulated cell death (RCD) in tumor cells and tissues by employing one or more Hsp90 inhibitors in combination with nanosecond pulsed electric fields (nsPEFs).

In some embodiments, drugs that inhibit the specific chaperone protein Hsp90 are employed in combination with nsPEFs to induce regulated cell death (RCD) in tumors and tissues, thereby enabling elimination of cancer cells. In these embodiments, cancer cells express elevated levels of Hsp90 as means to support neoplastic growth. Further to these embodiments, Hsp90 inhibitors in combination with nsPEF are employed at low non-toxic concentrations, thereby reducing the side-effects associated with aforementioned drugs. Still further to these embodiments, nsPEFs are employed at lower electric fields and/or with fewer number of pulses than when nsPEFs are employed alone. In these embodiments, the mechanism by which nsPEFs and Hsp90 inhibitors act upon cancer cells are different, thereby combining these treatments results in a synergistic effect. Further to these embodiments, this synergistic effect significantly eliminates cancer cells more effectively and with fewer side-effects than when either treatment is employed alone.

In some embodiments, the effect of Hsp90 inhibitors is selective and specific only affecting cancer cells. In these embodiments, even though Hsp90 is abundant in both normal and cancer cells, it is most often in a “sedate” state in normal cells, whereas it is often over-expressed in many cancers in an “activated” state with high affinity for ATP (100-fold). Further to these embodiments, the aforementioned activated state causes Hsp90 inhibitors to bind more tightly to its target in cancer cells compared to normal cells, thereby providing selectivity for effects of Hsp90 inhibitors on cancer versus normal tissue. Still further to these embodiments, nsPEFs are also selective and specific for cancer cell treatment based on the placement of tumors within the applied electric field. In these embodiments, during nsPEF treatments, normal cells outside the electrodes are not exposed to high electric fields. Further to these embodiments, the combination of both treatments (e.g., Hsp90 inhibitors and nsPEF) provide even greater specificity for targeting tumor cells over normal cells. Still further to these embodiments, the aforementioned treatment method employing a combination of Hsp90 inhibitors and nsPEFs can be used for treatment of tumors of different types, sizes, and at different tumor stages. In an example, the aforementioned treatment method employing a combination of Hsp90 inhibitors and nsPEFs is used for treating abnormal or benign growths, such as, for example in breast (adenosis, fibroadenomas mastitis) and lungs (harmartoma, papilloma), among others.

In some embodiments, Hsp90 inhibitors employed in combination with nsPEFs include 17-AAG, 17-DMAG, retaspimycin, macbecin, CNF-2024, CNF-1010, AT-13387, PF-04928473, STA-9090, AUY922, IPI-504, IPI-493, CCT018159, VER-49009, among others. In these embodiments, other means for inhibiting Hsp90, other Hsp90 inhibitors currently available, and gene-targeting approaches (e.g., siRNA, shRNA, CRISP against Hsp90, dominant negative or otherwise mutated Hsp90, and the like) can also be employed. Further to these embodiments, the electric pulses employed to generate nsPEFs employ a pulse duration from about 1 ns to about 1000 ns with amplitudes ranging from about 0.01 kV to about 300 kV. In an example, a single 600 ns pulse with an amplitude range from about 0 to about 80 kV/cm is employed.

In some embodiments, Hsp90 inhibitors inhibit HIF1a, which promotes expression of VEGF and vascularization of tumors. In these embodiments, nsPEFs also inhibit expression of VEGF as well as other endothelial cell markers, such as, for example CD31, CD35 and CD105, among others. Further to these embodiments, the combination of Hsp90 inhibitors and nsPEFs exhibit a synergistic effect to prevent vascularization in addition to the synergy of anti-tumor activities.

In some embodiments, the combination of Hsp90 inhibitor and nsPEF is administered alone as the sole therapeutic agent. In other embodiments, the combination of Hsp90 inhibitor and nsPEF is employed in combination with one or more additional therapies, such as, for example radiation therapy, electrotherapy, and immunotherapy, among others. In further embodiments, other anti-cancer drugs can be employed in combination with Hsp90 inhibitors and nsPEFs. In these embodiments, Hsp90 inhibitors can be administered using different routes of administration, such as, for example intravenous administration (IV), oral administration, intratumoral or intraperitoneal injection, and the like.

In some embodiments, Hsp90 inhibitors are delivered to the patient to sensitize tumor cells prior the application of nsPEFs. In other embodiments, nsPEFs are first applied to cancer cells or tumors to induce RCD. In these embodiments and after the application of nsPEFs, Hsp90 inhibitors are administered to the patient to eliminate remaining cancer cells after nsPEFs treatment. Further to these embodiments, nsPEFs act as well as a sensitizer for the subsequently treatment with Hsp90 inhibitors.

Reference will now be made to specific examples illustrating the use of nsPEFs in combination with Hsp90 inhibitors to induce RCD in tumor cells for the elimination of cancer. It is to be understood that the examples are provided to illustrate preferred embodiments and that no limitation of the scope of the disclosure is intended thereby.

### EXAMPLES

In some embodiments, inhibition of several possible Hsp90-regulated mechanisms can account for Hsp90 inhibitors anti-tumor activity. In these embodiments, Hsp90 functions as a molecular chaperone, thereby inhibition can lead to misfolding of client proteins. Further to these embodiments, Hsp90 protein acts as an anti-apoptotic factor, thereby inhibition can overcome evasion of apoptosis. Still further to these embodiments, Hsp90 inhibitors stimulate proteasomal degradation of client proteins, which can have anti-tumor consequences. In these embodiments, in the apoptosis pathway, Hsp90 possesses a dual effect on cell survival by both negatively regulating apoptotic protease activating factor 1 (APAF-1), which is required for caspase-9 activation, and by supporting AKT activation resulting in phosphorylation of Bcl-2 Associated Death (BAD), which then binds to 14-3-3 becoming unavailable for inducing cytochrome c release from mitochondria and apoptosis induction. Further to these embodiments, AKT also induces proliferation by promoting progression through the cell cycle by controlling G1/S and G2/M transitions.

In some embodiments, inhibition of Hsp90 by employing Hsp90 inhibitors abrogates aforementioned mechanisms and induces RCD, whereas NsPEF-induced cell death includes both caspase-dependent and caspase-independent RCD; however, nsPEF-induced mechanisms of cell death are cell type-specific and can be due to differential expression of proteins that regulate cell death. In these embodiments, nsPEFs induce cell death by mitochondrial-mediated mechanisms; however, in E4 squamous cell carcinoma, active caspase could be observed in the absence of cytochrome c release, thereby suggesting at least some degree of mitochondria-independent activation of caspases in that cell line.

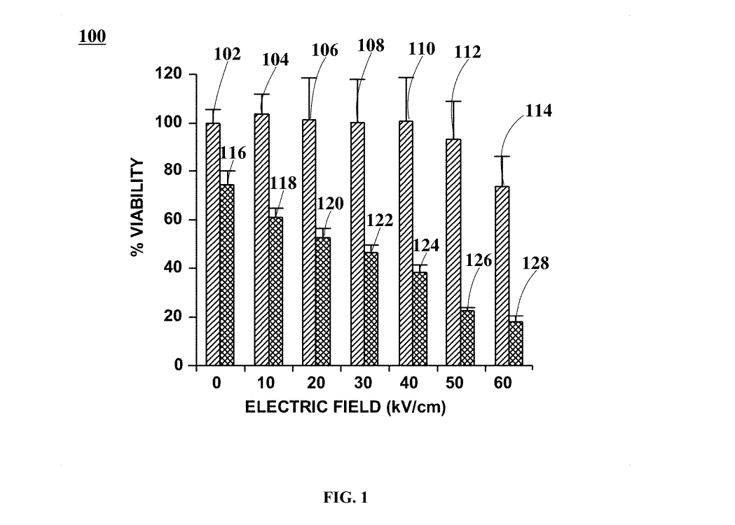

FIG. 1 is a graphical representation illustrating a synergistic induction of cell death in N1-S1 rat HCC cells employing nanosecond pulsed electric fields (nsPEFs) and Hsp90 inhibitor (e.g., 17-AAG), according to an embodiment. In FIG. 1, cell viability graph 100 includes: no treatment effect on viability bar 102; nsPEF effect on viability bars 104, 106, 108, 110, 112, and 114; Hsp90 inhibitor effect on viability bar 116; and combination of nsPEF and Hsp90 inhibitor effects on viability bars 118, 120, 122, 124, 126, and 128.

In some embodiments, control bars 102, 104, 106, 108, 110, 112, and 114 illustrate the percentage of cell viability associated with N1-S1 rat HCC cells that have been only exposed to nsPEFs and not to Hsp90 inhibitor. In these embodiments, treatment bar 116 illustrates the percentage of cell viability associated with N1-S1 rat HCC cells that have only been treated with Hsp90 inhibitor and not with nsPEFs. Further to these embodiments, treatment bars 118, 120, 122, 124, 126, and 128 illustrate the percentage of cell viability associated with N1-S1 rat HCC cells that have been treated with Hsp90 inhibitor (e.g., 17-AAG) and nsPEFs.

In some embodiments, N1-S1 rat HCC cells are exposed to an inhibitor of Hsp90 and then exposed to nsPEFs to cause cell death. In these embodiments, using the aforementioned methodology the cells mortality is greater than when either Hsp90 inhibitor or nsPEFs are employed alone. In an example, N1-S1 rat HCC cells are treated with 0.3 μM 17-AAG and incubated for 24 hours. In this example, after the 24 hours incubation period, the N1-S1 rat HCC cells are treated with a single 600 ns pulse with increasing electric field intensities (from about 10 kV/cm to about 60 kV/cm). Further to this example, cell viability is determined employing CellTiter-Glo (available from Promega, Madison, Wis.) 24 hours after pulsing.

In some embodiments and referring to FIG. 1, when employing nsPEFs alone and applying increasing electric fields (from about 10 kV/cm to about 60 kV/cm), only a small reduction (from about 0 to about 25%) in cell viability percentage is obtained (bars 104, 106, 108, 110, 112, and 114). In these embodiments, when employing 0.3 μM 17-AAG alone (bar 116) only about 25% of cell viability reduction is obtained. Further to these embodiments, the combination of both treatments (bars 118, 120, 122, 124, 126, and 128) exhibits cell viability percentage reductions from about 40% to about 80%. Still further to these embodiments, the combination of 17-AAG with nsPEFs induces a synergistic response significantly greater than the sum of the two treatments when employed alone. In these embodiments, the combination of 0.3 μM 17-AAG and nsPEFs having electric field amplitudes from about 10 kV/cm to about 60 kV/cm caused a synergy in cell death of about 1.6 to about 2.7 times the sum of the two treatments employed alone.

In some embodiments, there is a significantly higher decrease in cell viability compared to cells that were either treated with nsPEFs alone or with 17-AAG alone (e.g., 0 kV/cm). In these embodiments, inhibition of Hsp90 with 17-AAG sensitizes cells to nsPEFs thereby allowing lower electric fields to have a greater impact on induction of RCD. Further to these embodiments, test results demonstrate that an Hsp90 inhibitor in combination with nsPEFs induces cell death that is significantly greater than cell death induced by either Hsp90 inhibitor or nsPEFs alone or their algebraic sum.

In some embodiments, a single 600 ns pulse of 50 kV/cm did not exhibit a statistical effect on rat HCC cell death. In other embodiments, when a substantially similar nsPEF pulse is employed on cells pre-treated with 0.3 μM 17-AAG, 80% of the cells are killed. Further to these embodiments, this synergism demonstrates that cell death induced by Hsp90 inhibitors and nsPEFs possess different mechanisms of action.

In some embodiments, given that 17-AAG and nsPEFs act synergistically to induce RCD, it is expected that they perform this function by different cell death mechanisms. In these embodiments, the 17-AAG may induce apoptosis by mitochondria-mediated mechanism(s) that leads to caspase activation. Further to these embodiments, nsPEFs function by a different mechanism(s), such as, for example affecting proteins that regulates the mitochondria membrane potential (ΔΨm).

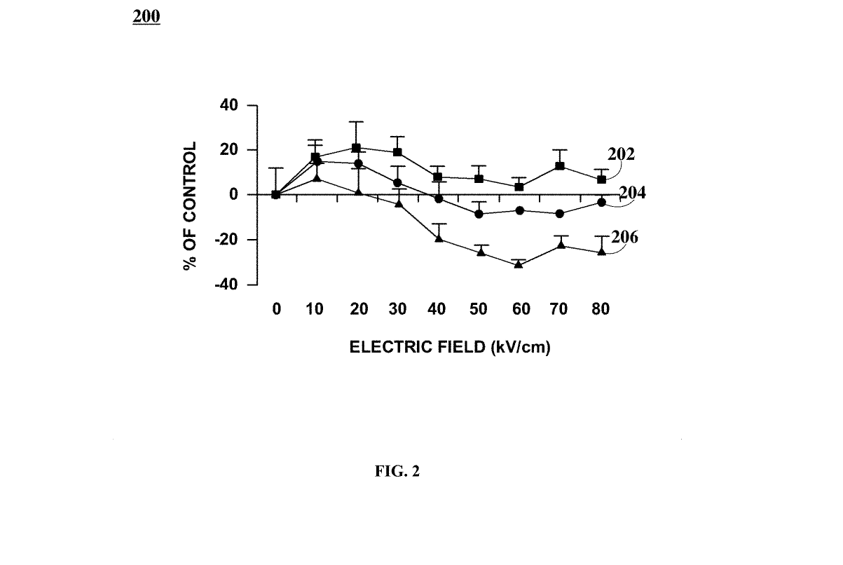

FIG. 2 is a graphical representation illustrating effects of the nsPEFs on caspase 3/7 catalytic activity as compared to a control, according to an embodiment. In FIG. 2, caspase activity graph 200 includes catalytic activity lines 202, 204, and 206.

In FIG. 2, catalytic activity lines 202, 204, and 206 illustrate the percentage of caspase 3/7 catalytic activity of rat N1-S1 HCC cells two (2) hours, four (4) hours, and six (6) hours after the application of a single 600 ns pulse at different electric field intensities, respectively. In an example, caspase 3/7 catalytic activity is measured employing Caspase-Glo (available from Promega, Madison, Wis.).

In some embodiments, increases in caspase 3/7 activity at low electric fields (10-30 kV/cm) are modest (from about 10% to about 20%) and no increases in caspase 3/7 activity are observed with higher electric fields (from about 30 kV/cm to about 80 kV/cm) at any of the measurement times (catalytic activity lines 202, 204, and 206). In these embodiments, no increases in caspase 3/7 activity are observed six (6) hours after pulsing (catalytic activity line 206). Further to these embodiments, decreases in caspase activity below basal untreated control levels are observed in electric fields intensities greater than 40 kV/cm. In summary, nsPEFs do not increase caspase 3/7 catalytic activity in N1-S1 rat HCC cells that would result in significant loss of cell viability.

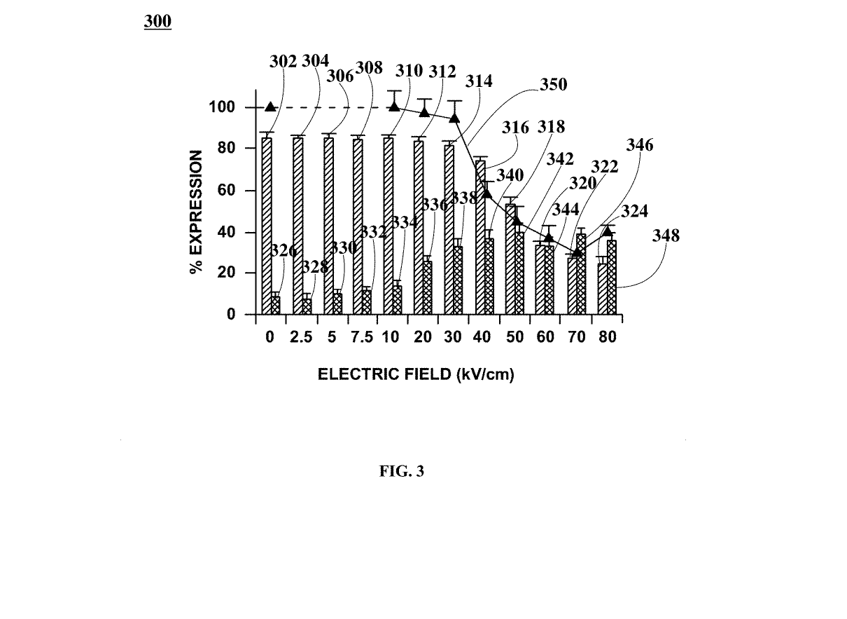

FIG. 3 is a graphical representation illustrating effects of the nsPEFs on mitochondria membrane potential (ΔΨm), Ca²⁺ influx, and cell viability in N1-S1 HCC cells, according to an embodiment. In FIG. 3, results graph 300 includes: mitochondria membrane potential bars 302, 304, 306, 308, 310, 312, 314, 316, 318, 320, 322, and 324; Ca²⁺ influx bars 326, 328, 330, 332, 334, 336, 338, 340, 342, 344, 346, and 348; and viability line 350.

In FIG. 3, mitochondria membrane potential bars 302, 304, 306, 308, 310, 312, 314, 316, 318, 320, 322, and 324 represent the change in ΔΨm 10 minutes after the application of a single 600 ns pulse at different electric field amplitudes. In FIG. 3, Ca²⁺ influx bars 326, 328, 330, 332, 334, 336, 338, 340, 342, 344, 346, and 348 represent the change in Ca²⁺influx 10 minutes after the application of a single 600 ns pulse at different electric field amplitudes. In FIG. 3, viability line 350 represents the change in cell viability 24 hours after the application of a single 600 ns pulse at different electric field amplitudes.

In an example, mitochondria membrane potential (ΔΨm) and Ca²⁺ influx are determined by employing Tetramethylrhodamine, ethyl ester (TMRE) Assay Kit and green-fluorescent calcium indicator, Fluo-4, respectively, 10 minutes after pulsing. In this example, cell viability is determined employing CellTiter-Glo (available from Promega, Madison, Wis.) 24 hours after pulsing.

In some embodiments, a single 600 ns pulse with electric field intensities greater than or equal to 40 kV/cm dissipates ΔΨm (bars 316, 318, 320, 322, and 324) and induces RCD (viability line 350). In these embodiments, nsPEF causes permeabilization of the plasma membrane thereby allowing greater influxes of extracellular Ca²⁺ at electric field intensities greater than or equal to 20 kV/cm (bars 336, 338, 340, 342, 344, 346, and 348). In summary, nsPEFs induce dissipation of ΔΨm and reduce cell viability by inducing apoptosis in a caspase-independent mechanism.

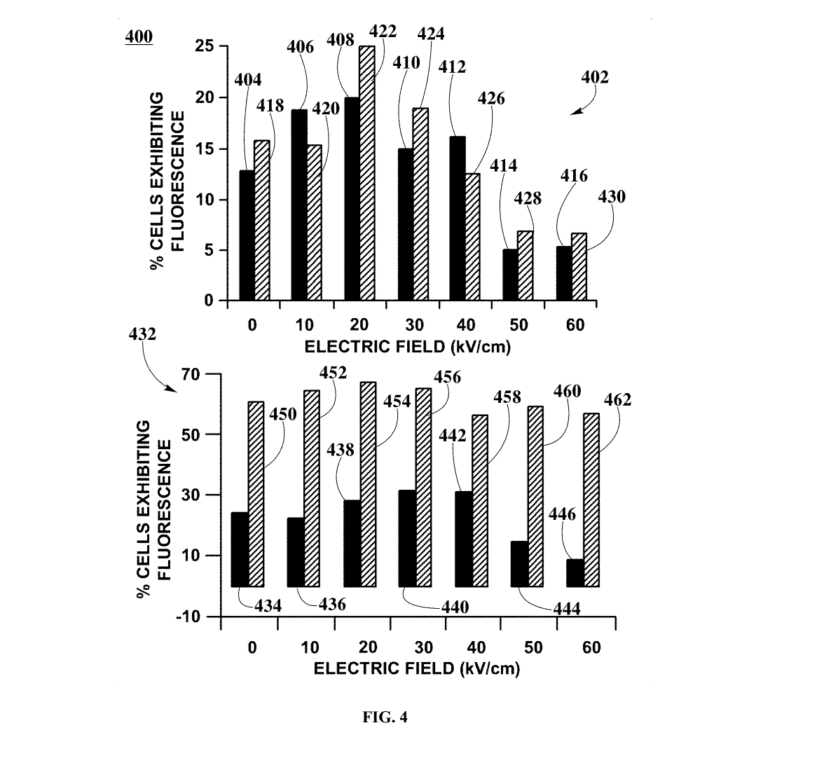

FIG. 4 is a graphical representation illustrating effects of the Hsp90 inhibitor (e.g., 17-AAG) on caspase 3/7 catalytic activity in N1-S1 cells at different incubation times, according to an embodiment. In FIG. 4, results graph 400 includes bar graph 402 and bar graph 432. In FIG. 4, bar graph 402 includes control bars 404, 406, 408, 410, 412, 414, and 416, and 4-hours incubation bars 418, 420, 422, 424, 426, 428, and 430. In FIG. 4, bar graph 432 includes control bars 434, 436, 438, 440, 442, 444, and 446, and 16-h incubation bars 450, 452, 454, 456, 458, 460, and 462.

In some embodiments, bar graph 402 represents the percentage change in caspase 3/7 catalytic activity after four (4) hours of incubation with 0.3 μM 17-AAG followed by the application of a single 600 ns pulse at different electric field intensities (from 0 to about 60 kV/cm). In these embodiments, bar graph 432 represents the percentage change in cell viability after 16 hours of incubation with 0.3 μM 17-AAG followed by the application of a single 600 ns pulse at different electric field intensities (from 0 to about 60 kV/cm).

In some embodiments, N1-S1 cells were incubated with or without 17-AAG for 4 hours or 16 hours to determine a synergistic activation of caspase 3/7 with 17-AAG and nsPEFs. In these embodiments, after the incubation period, N1-S1 cells were then treated with nsPEFs with increasing electric fields (one 600 ns pulse) and assayed for caspase 3/7 catalytic activity two (2) hours after pulsing. In an example, caspase 3/7 catalytic activity is measured employing Caspase-Glo (available from Promega, Madison, Wis.) 2 hours after pulsing.

In some embodiments, no significant change in caspase activity is observed after 4 hours of incubation (bar graph 402) when employing 17-AAG alone (bar 418). In these embodiments, nsPEFs cause modest increases in caspase activity at lower electric fields (bars 406-412) as previously demonstrated in FIG. 2, above. Further to these embodiments, there is no effect of 17-AAG on the modest change in caspase activity at lower electric fields (10-40 kV/cm, bars 420, 422, 424, and 426). Still further to these embodiments, there is no effect of 17-AAG on the modest change in catalytic activity at higher electric fields (50-60 kV/cm, bars 428 and 430).

In some embodiments, after 16 h of incubation with 17-AAG, there are significant increases in caspase activity that is unaffected by nsPEFs. In these embodiments, 17-AAG causes increases in caspase 3/7 activity between the 4^{th} and 16^{th} hour, while nsPEFs cause only modest changes, primarily decrease in activity as illustrated in FIG. 2. Further to these embodiments, there is no synergistic effects on caspase 3/7 activity when the two treatments are included together, thereby indicating that nsPEFs and Hsp90 inhibitors induced RCD by different mechanisms.

In summary, the combination of 17-AAG and nsPEF provide a synergistic induction of RCD by different mechanisms in N1-S1 HCC cells. Further, 17-AAG increases caspase 3/7 activity and consequently apoptotic cell death, whereas nsPEFs induce influxes of Ca²⁺, dissipation of ΔΨm, and RCD by a caspase-independent mechanism(s). Additionally, 17-AAG sensitizes cells to nsPEFs and the combination of both treatments is significantly improved than either treatment alone. Therefore, it is possible to employ low non-toxic doses of 17-AAG or other Hsp90 inhibitor and low “doses” of nsPEFs by decreasing electric field intensities or decreasing the number of pulses employed for treatment of cancer cells.

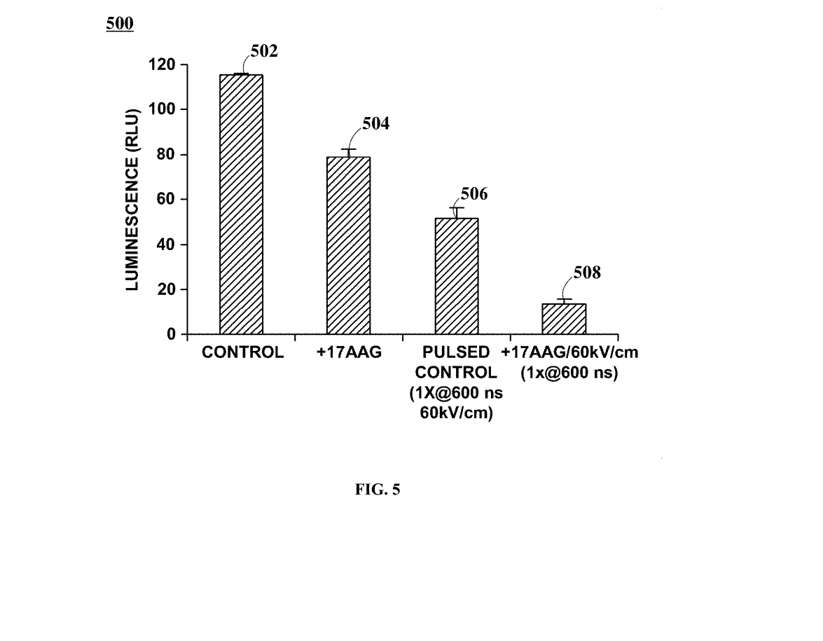

FIG. 5 is a graphical representation illustrating effects on cell viability of an Hsp90 inhibitor (e.g., 17-AAG) in combination with an nsPEF, according to an embodiment. In FIG. 5, cell viability graph 500 includes bars 502, 504, 506, and 508.

In some embodiments, bar 502 illustrates the percentage of cell viability for a control sample where none of the treatments are employed. In these embodiments, bar 504 illustrates the percentage of cell viability when employing an Hsp90 inhibitor at a concentration of 0.3 μM (e.g., 17-AAG) without nsPEFs. Further to these embodiments, bar 506 illustrates the percentage of cell viability when employing only a single 600 nsPEF of 60 kV/cm. Still further to these embodiments, bar 508 illustrates the percentage of cell viability when employing both treatments (0.3 μM 17-AAG and single 600 ns pulse of 60 kV/cm). In an example, N1-S1 HCC cells are employed for the cell viability test.

In some embodiments, the combination of 0.3 μM 17-AAG and a single 600 ns of 60 kV (bar 508) exhibit the lowest percentage of cell viability attaining only about 17%. In these embodiments, the pulsed control (bar 506) employing nsPEF alone exhibits around 45% of cell viability, followed by 0.3 μM 17-AAG employed alone (bar 504) and the control sample (bar 502) where no treatment is applied. Further to these embodiments, the combination of nsPEFs and Hsp90 inhibitors (bar 508) exhibits the lower percentage of cell viability when compare to each treatment employed alone, thereby indicating a synergistic effect.

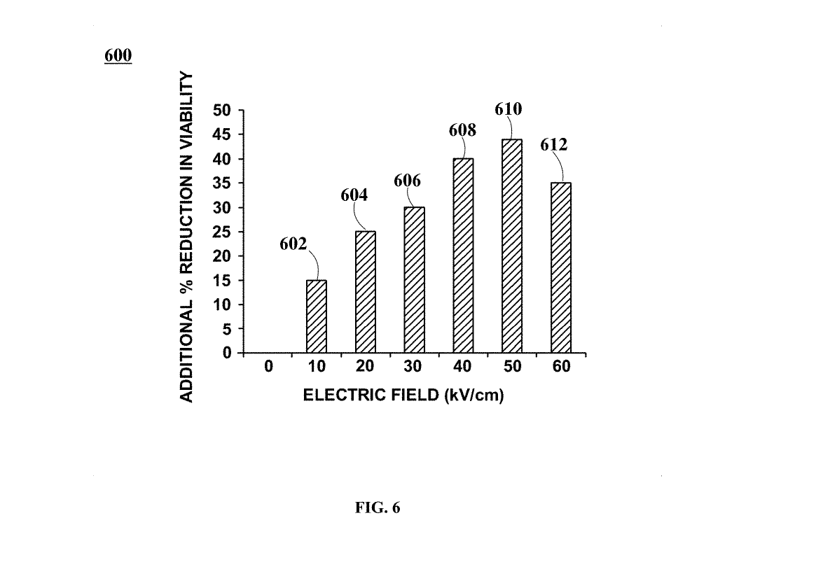

FIG. 6 is a graphical representation illustrating an additional percentage reduction in cell viability when employing an Hsp90 inhibitor at a concentration of about 0.3 μM (e.g., 17-AAG) in combination with nsPEFs at different electric field amplitudes, according to an embodiment. In FIG. 6, cell viability graph 600 includes bars 602, 604, 606, 608, 610, and 612.

In some embodiments, there is an increase in the additional percentage of cell viability reduction attained when increasing the amplitude of the electric field. In these embodiments, there is about 45% (bar 610) additional increase in the reduction of cell viability at an electric field amplitude of about 50 kV/cm.

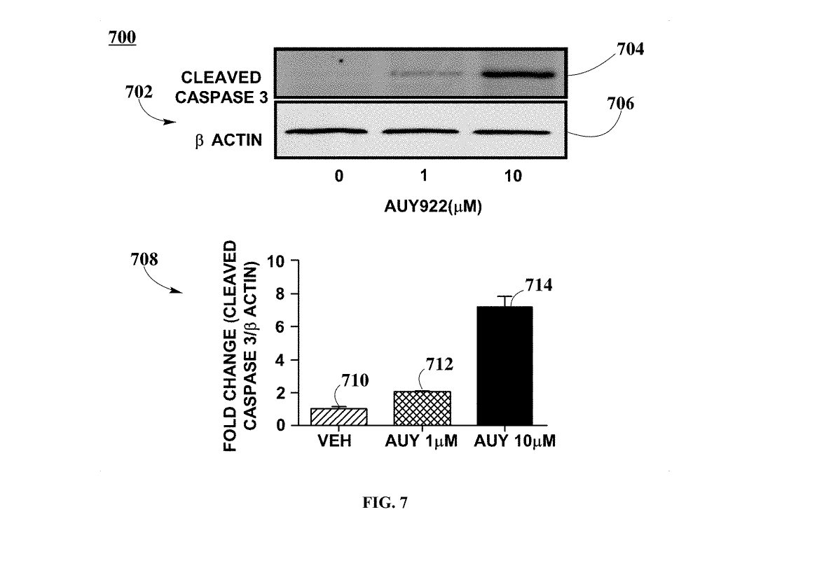

FIG. 7 is a graphical representation illustrating western blot test results for cleaved caspase 3 when employing an Hsp90 inhibitor at different concentrations, according to an embodiment. In FIG. 7, western blot test results 700 include stained bands 702 and cleaved caspase 3 fold change graph 708. In FIG. 7, stained bands 702 include cleaved caspase 3 bands 704 and β-actin bands 706. In FIG. 7, cleaved caspase 3 fold change graph 708 includes bars 710, 712, and 714. In an example, the western blot data is quantified using a LI-COR Odyssey CLx imaging system.

In FIG. 7, stained bands 702 is a photographic representation depicting an expression of cleaved caspase 3 as a function of concentration of an Hsp90 inhibitor (e.g., AUY922). In FIG. 7, cleaved caspase 3 fold change graph 708 is a graphical representation illustrating fold changes of caspase 3 with respect to β-actin.

In some embodiments, cleaved caspase 3 bands 704 illustrate the expression of cleaved caspase 3 at different concentrations of AUY922. In these embodiments, β-actin bands 706 illustrate bands for β-actin as a loading control. Further to these embodiments, cleaved caspase bands 704 exhibit the greatest expression of cleaved caspase 3 when the concentration of AUY922 is 10 μM.

In some embodiments, bars 710, 712, and 714 illustrate fold change ratios of cleaved caspase 3 with respect to (3-actin as a loading control at different concentrations of Hsp90 inhibitor (e.g., AUY922). In these embodiments, at a concentration of about 10 μM of AUY922, the ratio of caspase 3 to β-actin is about 7 (bar 714), whereas at a concentration of 1 μM of AUY922, the fold change is about 2 (bar 712). Further to these embodiments, when no AUY922 is employed, the aforementioned ratio is about 1 (bar 710). In these embodiments, these test results demonstrate that the mechanism by which Hsp90 inhibitors (e.g., AUY922) act to induce RCD is caspase-dependent.

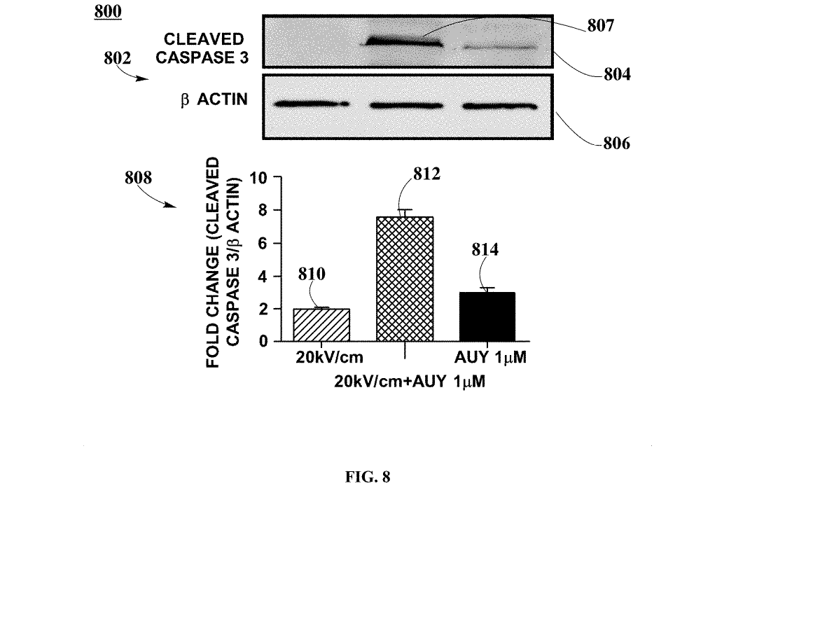

FIG. 8 is a graphical representation illustrating western blot test results for cleaved caspase 3 when employing an Hsp90 inhibitor alone, nsPEF alone, or a combination of both treatments, according to an embodiment. In FIG. 8, western blot test results 800 include stained bands 802 and caspase 3 fold change graph 808. In FIG. 8, stained bands 802 include cleaved caspase 3 bands 804, β-actin bands 806, and caspase expression 807. In FIG. 8, caspase 3-fold change graph 808 includes bars 810, 812, and 814. In an example, the western blot data is quantified using LI-COR Odyssey CLx imaging system.

In some embodiments, stained bands 802 is a photographic representation depicting an expression of cleaved caspase 3 as a function of the treatment employed. In these embodiments, caspase 3 fold change graph 808 is a graphical representation illustrating fold changes of caspase 3 with respect to β-actin.

In some embodiments, cleaved caspase 3 bands 804 illustrate the expression of cleaved caspase 3 as a function of the treatment employed. In these embodiments, β-actin bands 806 illustrates the bands for β-actin as a loading control. Further to these embodiments, caspase expression 807 illustrates the expression of cleaved caspase 3 after treatment with an Hsp90 inhibitor and the application of an nsPEF. In these embodiments, the expression of cleaved caspase 3 (caspase expression 807) is higher when employing the combination of an Hsp90 inhibitor and an nsPEF of 600 ns with an electric field of 20 kV/cm.

In some embodiments, bars 810, 812, and 814 illustrate fold change ratios of cleaved caspase 3 with respect to a β-actin loading control employing different treatment options including nsPEF alone (bar 810), a combination of nsPEF and AUY922 (bar 812), and AUY922 alone (bar 814) at a concentration of 1 μM. In these embodiments, when employing the combination of a Hsp90 inhibitor at a concentration of 1 μM and a nsPEF of 600 ns with an electric field of 20 kV/cm, the ratio of caspase 3 to β-actin is about 7 (bar 812), whereas when employing nsPEF and AUY922 by themselves the fold change is about 2 (bar 810) and 3 (bar 814), respectively. Further to these embodiments, these test results demonstrate the synergy between Hsp90 inhibitor and nsPEF to induce RCD.

In summary, the combination of Hsp90 inhibitors and nsPEFs provide a synergistic induction of RCD by different mechanisms in cancer cells. Further, Hsp90 inhibitors increase caspase 3/7 activity and consequently apoptotic cell death, whereas nsPEF induces influxes of Ca²⁺, dissipation of ΔΨm, and RCD by a caspase-independent mechanism(s). Additionally, Hsp90 inhibitors sensitize cells to nsPEFs and the combination of both treatments is a significant improvement over either treatment employed alone. Therefore, it is possible to employ low non-toxic doses of 17-AAG or other Hsp90 inhibitor and low “doses” of nsPEFs by decreasing electric field intensities or decreasing the number of pulses for treatment of cancer cells. The combination of nsPEFs and an Hsp90 inhibitor can be employed to effectively kill cancer cells while exhibiting a reduction of the side-effects associated with both treatments.

While various embodiments of the present disclosure have been described above, it should be understood that they have been presented by way of example only, and not limitation. Numerous changes to the disclosed embodiments can be made in accordance with the disclosure herein without departing from the spirit or scope of the disclosure. Thus, the breadth and scope of the present disclosure should not be limited by any of the above described embodiments.

Rather, the scope of the disclosure should be defined in accordance with the following claims and their equivalents.

Although the present disclosure has been illustrated and described with respect to one or more implementations, equivalent alterations and modifications can occur to others skilled in the art upon the reading and understanding of this specification and the drawings. In addition, while a particular feature of the disclosure may have been disclosed with respect to only one of several implementations, such feature may be combined with one or more other features of the other implementations as may be desired and advantageous for any given or particular application.

The terminology used herein is for the purpose of describing particular embodiments only and is not intended to be limiting of the invention. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”0

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

What is claimed is:

1. A method of inducing regulated cell death in a target tissue or target cell population, comprising:
inhibiting Hsp90 in said target tissue or target cell population; and
applying an electrical stimulation to said target tissue or target cell population, said electrical stimulation comprising one or more electric pulses having a pulse duration from about 1 nanoseconds to about 1,000 nanoseconds, and further having pulse amplitudes ranging from about 0.01 kV to about 300 kV,
in a synergistic manner effective to induce tissue or cell death in said target tissue or target cell population near an applied electric field created by said electrical stimulation.

2. The method of claim 1, wherein the target tissue comprises a tumor.

3. The method of claim 1, wherein the target cell population comprises cancer cells.

4. The method of claim 1, wherein the levels of Hsp90 within the target tissue or target cell population is over-expressed.

5. The method of claim 1, wherein the step of inhibiting comprises administering at least one Hsp90 inhibitor to said target tissue or target cell population.

6. The method of claim 4, wherein said at least one Hsp90 inhibitor is selected from the group consisting of 17-AAG, 17-DMAG, retaspimycin, macbecin, CNF-2024, CNF-1010, AT-13387, PF-04928473, STA-9090, AUY922, IPI-504, IPI-493, CCT018159, and VER-49009.

7. The method of claim 4, wherein said at least one Hsp90 inhibitor is administered by intravenous administration, oral administration, intratumoral or intraperitoneal injection.

8. The method of claim 1, wherein the step of inhibiting Hsp90 employs a gene-targeted expression or promotion technique comprising siRNA, ShRNA, CRISP against Hsp90, or dominant negative or mutated Hsp90.

9. The method of claim 1, further including preventing vascularization in said target tissue or target cell population.

10. The method of claim 1, further including inhibiting expression of VEGF, CD31, CD36 or CD105.

11. The method of claim 1, wherein the step of inhibiting Hsp90 occurs before the step of applying electrical stimulation.

12. The method of claim 1, wherein the step of inhibiting Hsp90 occurs after the step of applying electrical stimulation.

13. The method of claim 1, further including the step of administering an additional treatment technique comprising radiation therapy, electrotherapy, or immunotherapy.

14. The method of claim 1, further including administering an anti-cancer drug to said target tissue or target cell population.

15. The method of claim 1, wherein the step of applying electrical stimulation comprises placing electrodes into said target tissue or said target cell population in proximity to cancer cells.

16. The method of claim 1, wherein said at least one electric pulse comprises a pulse duration of about 600 ns.

17. The method of claim 1, wherein said at least one electric pulse comprises an amplitude of between about 10 kV/cm to about 80 kV/cm.
